(19) United States
(12) Patent Application Publication
(10) Pub. No.: US 2008/0009652 A1
(43) Pub. Date: Jan. 10, 2008

(54) Process of Making Butyric Acid

(75) Inventor: Zhiping Shan, The Woodlands, TX (US)

Correspondence Address: Legal Department;Huntsman Corporation, 10003 Woodloch Forest Drive, The Woodlands, TX, 77380, US

(73) Assignee: Huntsman Petrochemical Corporation, The Woodlands, TX (US)

(21) Appl. No.: 11/773,498

(22) Filed: Jul. 5, 2007

Related U.S. Application Data
(60) Provisional application No. 60/818,686, filed on Jul. 5, 2006.

(51) Int. Cl. C07C 51/347

(52) U.S. Cl. 562/606

(57) ABSTRACT

Processes for forming butyric acid are provided. In one process, maleic anhydride is formed by oxidizing a hydrocarbon containing gas. The maleic anhydride is then hydrogenated in the presence of a hydrogenation catalyst to form butyric acid. The selectivity of maleic anhydride to butyric acid is at least about 35 molar percent.

### CROSS-REFERENCE TO RELATED APPLICATIONS

This application claims the benefit of U.S. Provisional Application No. 60/818,686 filed on Jul. 5, 2006.

### BACKGROUND OF THE INVENTION

1. Field of the Invention

This invention relates, generally, to the production of butyric acid, and more specifically to the production of butyric acid by hydrogenating maleic anhydride in the presence of a hydrogenation catalyst.

2. Background of the Invention

Butyric acid (C₄H₈O₂) has been used in the chemical, food, and pharmaceutical industries. In one application, butyric acid has been used to enhance butter-like notes for food flavors. In another application, esters of butyric acid have been used to increase fruit fragrance, and as aromatic compounds for the production of perfumes. Butyric acid may also be used as a raw material for the production of biodegradable polymers in the form of β-hydroxybutyrate.

Various methods of producing butyric acid have included oxidation of n-butyraldehyde, which was obtained from propylene hydroformylation (oxo reaction) under relatively high pressure. This process, however, has several drawbacks include the expensive propylene raw material, and the relevantly low selectivity in the production n-butyraldehyde. Additionally, this process typically uses a homogeneous catalyst during hydroformylation. It is difficult to separate homogeneous catalyst from the process.

Another method of producing butyric acid includes fermentation of a sugar or starch directly into butyric acid or indirectly to butanol, which is followed by oxidation. The fermentation process, however, yields relatively low productivity and concentration of the final product. Fermentation additionally requires a relatively complicated and expensive isolation process.

Maleic anhydride hydrogenation (using conventional hydrogenation catalysts) typically produces compounds such as 1,4-butanediol (BDO), γ-butyrolacetone, succinic acid and succinic anhydride. For example, U.S. application Ser. No. 10/883,106 to Bhattacharyya discloses a process for catalytically hydrogenating a hydrogenatable precursor in contact with a hydrogen-containing gas and a hydrogenation catalyst comprising one or more active hydrogenation catalyst components on a support comprising titanium dioxide in the rutile crystalline phase to produce 1,4-butanediol and, optionally, gamma-butyrolactone and/or tetrahydrofuran. Maleic anhydride hydrogenation does not typically produce butyric acid as a direct product.

Accordingly, there is a need for an efficient, simple, and cost-effective process for producing butyric acid.

### BRIEF SUMMARY OF SOME OF THE PREFERRED EMBODIMENTS

For the purposes of this disclosure, the term “selectivity” means the actual mole percent of a reactant that is chemically altered into a desired product.

For the purposes of this disclosure, the term “conversion” means the actual mole percent of a reactant that is chemically altered.

For the purposes of this disclosure, the term “yield” is calculated by multiplying the selectively and conversion.

In an embodiment, the inventions disclosed herein relate to a process for making butyric acid. The process includes combining a maleic anhydride, a hydrogen containing gas, and a hydrogenation catalyst. The selectivity of maleic anhydride to butyric acid is at least about 35 molar percent.

In another embodiment, the inventions disclosed herein relate to a process for making butyric acid. The process includes combining one or more hydrocarbons and an oxygen gas selected from the group consisting of dioxygen gas, oxygen containing gas, and mixtures thereof to form maleic anhydride. The process further includes combining the maleic anhydride, a hydrogen containing gas, and a hydrogenation catalyst. The selectivity of maleic anhydride to butyric acid is at least about 35 molar percent.

In a still further embodiment, the inventions disclosed herein relate to a process of comprising: (a) combining one or more hydrocarbons and an oxygen gas selected from the group consisting of dioxygen, an oxygen containing gas, and mixtures thereof to form maleic anhydride; (b) selectively absorbing the maleic anhydride in an organic solvent selected from the group consisting of dibutyl phthalate (DBP), diisobutyl hexahydrophthalate, diisobutyl tetrahydrophthalate, dibutyl hexahydrophthalate, and dibutyl tetrahydrophthalate to form an absorption liquid; (c) stripping the absorption liquid into a crude maleic anhydride, and optionally purifying the crude maleic anhydride into a maleic anhydride product; (d) combining the maleic anhydride product and a second organic solvent selected from the group consisting of ethanol, propanol, isopropanol, isobutanol, ethyl acetate, and mixtures thereof to form a maleic anhydride solution, and optionally combining the crude maleic anhydride and a second organic solvent selected from the group consisting of ethanol, propanol, isopropanol, isobutanol, ethyl acetate, and mixtures thereof to form a maleic anhydride solution; (e) heating the maleic anhydride solution to at least about 100° C.; (f) combining the heated maleic anhydride solution, a hydrogen containing gas, and a hydrogenation catalyst to form a crude butyric acid, wherein the hydrogentation catalyst comprises at least one noble metal supported on at least one metal oxide, wherein the selectivity of maleic anhydride product to crude butyric acid is at least about 35 molar percent; and (g) purifying the crude butyric acid into a butyric acid product, wherein the butyric acid product is at least about 80 weight percent butyric acid.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### DETAILED DESCRIPTION OF THE PREFERRED EMBODIMENTS

In an embodiment, a process of making butyric acid comprises an oxidation step and a hydrogenation step.

### Oxidation Step

In the oxidation step, a hydrocarbon is contacted with dioxygen and/or an oxygen containing gas—such as air—to form maleic anhydride and possibly by-products. Preferably, an oxidation catalyst is used in the oxidation step to increase the selectively. The oxidation step may be carried out in any suitable reactor, and is preferably carried out in a fixed-bed reactor or a fluid-bed reactor.

The hydrocarbon preferably has at least four carbon atoms in a straight chain. Examples of suitable hydrocarbons include n-butane, n-butene, benzene, and mixtures thereof.

The oxidation catalyst preferably comprises vanadium, phosphorous, and oxygen, such as a vanadium phosphate oxide catalyst (“VPO catalyst”). Examples of VPO catalysts are described in U.S. Pat. No. 5,773,382, U.S. Pat. No. 6,107,234 and U.S. Pat. No. 6,812,351. The oxidation catalyst may further comprise promoters, activators or modifiers such as antimony, bismuth, boron, cerium, chromium, cobalt, copper, iron, lithium, molybdenum, nickel, niobium, silicon, tin, titanium, tungsten, uranium, zinc, zirconium, and mixtures thereof.

In an embodiment, wherein the oxidation step is carried out in fixed-bed reactor, the bed of the reactor is charged with catalyst. The reactant gases—for example hydrocarbon and an oxygen containing gas—are passed through the bed. In an embodiment, the reactant gas contains between about 1.5 to about 2.5 mole percent, alternatively between about 1.7 and 2.2 mole percent, and alternatively about 2.0 mole percent hydrocarbon and the balance oxygen gas mixture. In another embodiment, the gas in the reactor comprises between about 1.5 to about 2.5 mole percent, alternatively between about 1.7 and 2.2 mole percent, alternatively about 2.0 mole percent hydrocarbon and the balance an oxygen containing gas mixture, which includes inert gas.

The oxidation step is a generally exothermic process. Accordingly, the fixed bed may utilize a shell-tube reactor to remove reaction heat. In this embodiment, the catalyst is packed inside the tubes, and a cooling fluid flows through the shell side to remove heat generated inside the tubes. An example of a suitable cooling fluid is molten salt. Generally, the temperature inside the tubes range from about 350° C. to about 450° C., and hot spots may reach from about 450° C. to about 550° C.

In an embodiment, wherein the oxidation step is carried out in a fluid-bed reactor, the fluid bed is charged with catalyst. The reactant gases—for example hydrocarbon and an oxygen containing gas—are preferably separately fed into the fluidized bed. In an embodiment, the reactant gas contains between about 1.0 to about 10.0 molar percent, alternatively between about 2.0 and 8.0 molar percent, alternatively between about 3.0 to about 5.0 molar percent hydrocarbon and an oxygen containing gas. In another embodiment, the gas in the reactor comprises between about 1.0 to about 10.0 molar percent, alternatively between about 2.0 and 8.0 molar percent, alternatively between about 3.0 to about 5.0 molar percent hydrocarbon and an oxygen containing gas mixture, which includes inert gas. Reaction heat may be withdrawn by internal coils that preferably generate high pressure steam. Ordinarily, some catalyst becomes entrained in the effluent gases. A cyclone, or series of cyclones, may be used to recover the entrained catalyst. The catalyst recovered from the cyclone is preferably cooled, filtered, and returned to the reactor. In one embodiment, the fluid-bed reactor requires highly attrition-resistant catalyst such as a VPO catalyst coated with silica.

The gases exiting the reactor generally contain maleic anhydride together with by-products and inert gases. In an embodiment, the conversion of hydrocarbon to maleic anhydride ranges from about 70 to 90 mole percent, alternatively from about 75 to 85 mole percent, alternatively from about 80 to 83 mole percent. In an embodiment, the yield of maleic anhydride ranges from about 40 to about 70 mole percent, alternatively from about 45 to 65 mole percent, alternatively from about 50 to 58 mole percent. The by-products may include carbon monoxide, carbon dioxide, water vapor, acrylic acid, acetic acids, and the like. Maleic anhydride may be recovered by condensing the gases exiting the reactor at a temperature between the dew point of maleic anhydride and water. The dew point of both maleic anhydride and water will vary based on a number of factors, including pressure and concentration.

Maleic anhydride may be recovered using any suitable recovery system. Examples of such recovery systems are generally disclosed in U.S. Pat. Nos. 5,929,255, 6,120,654, and 6,090,245, which are each herein incorporated by reference in their entirety. A preferred method of recovery involves selectively absorbing maleic anhydride in a suitable organic solvent, and then stripping the maleic anhydride from the resulting absorption liquid to obtain a crude maleic anhydride product. The crude maleic anhydride may be further purified by distillation. Examples of suitable organic solvent include heavy esters such as phthalate ester and hydrophthalates that include for example dibutyl phthalate (DBP), diisobutyl hexahydrophthalate, diisobutyl tetrahydrophthalate, dibutyl hexahydrophthalate, dibutyl tetrahydrophthalate, and the like.

In an alternative recovery system water (e.g., instead of organic solvent) is used to absorb the remaining maleic anhydride in the scrubber tower and obtain a maleic acid solution. The solution is concentrated and dehydrated to obtain crude maleic anhydride.

### Hydrogenation Step

In the hydrogenation step, maleic anhydride is contacted with a hydrogen containing gas—such as hydrogen gas—to form butyric acid. Preferably, a hydrogenation catalyst is used in the hydrogenation step to increase the selectively. The hydrogenation may be carried out as either a liquid phase process or a gas phase process. Preferably, the hydrogenation step results in the direct production of butyric acid. This is in contrast to hydrogenation that results in the production of an intermediate such as gamma-butyrolactone, which must be synthesized into butyric acid. The preferable hydrogenation step has a selectivity of at least 35 molar percent, which may be contrasted with hydrogenation that results in the production of butyric acid as a byproduct.

The hydrogenation catalyst preferably comprises at least one noble metal and at least one metal oxide. Alternatively, the catalyst may comprise at least one transition metal and at least one metal oxide. Alternatively, the catalyst may comprise at least one noble metal, at least one transition metal and at least one metal oxide. Examples of suitable noble metals include gold, platinum, palladium, rhodium, ruthenium, silver, tantalum, mixtures thereof, and the like. Examples of suitable metal oxides include oxides of aluminum, chromium, iron, manganese, tin, titanium, vanadium, and zirconium, such as alumina oxide and titanium oxide, as well as mixtures thereof, and the like. Examples of suitable transition metals include chromium, cobalt, copper, hafnium, iron, magnesium, molybdenum, nickel, niobium, titanium, vanadium, zirconium, mixtures thereof, and the like.

A preferred hydrogenation catalyst for use in a liquid phase process comprises at least one noble metal supported on, i.e., chemically and/or physically bound to, at least one metal oxide, for example Pd/TiO₂. A preferred hydrogenation catalyst for use in a gas phase process comprises at least one noble metal and one transition metal supported on, i.e., chemically and/or physically bound to, at least one metal oxide, for example Cu—Pd/TiO₂/γ-Al₂O₃.

The hydrogenation catalyst preferably comprises noble metal in an amount less than about 5 weight percent, alternatively less than about 3 weight percent, alternatively in an amount ranging from about 0.2 to about 2.5 weight percent, and the balance metal oxide. The hydrogenation catalyst preferably comprises noble metal in an amount less than about 5 weight percent, alternatively less than about 3 weight percent, alternatively in an amount ranging from about 0.2 to about 2.5 weight percent, transition metal in an amount ranging from about 1 to about 30 weight percent, alternatively from about 1 to about 20 weight percent, alternatively from about 2 to about 15 weight percent, and the balance metal oxide.

In an embodiment, the hydrogenation step may be carried out in a liquid phase within a batch reactor, a continuous-flow reactor, or a semi-continuous flow reactor. In the liquid-phase batch reactor process, an organic solution of maleic anhydride is preferably prepared outside of the reactor. The solution is then fed into the batch reactor, which was pre-charged with hydrogenation catalyst. Alternatively, the organic solvent, maleic anhydride, and catalyst may be fed into the batch reactor at about the same time. The solution is heated to a predetermined temperature, and then a hydrogen containing gas is fed into the reactor under agitation. After a period of time, the processed is stopped. The contents of the reactor are removed, the butyric acid is separated from the catalyst, and purified. Suitable organic solvents generally include alcohols and esters, such as ethanol, propanol, isopropanol, isobutanol, ethyl acetate, mixtures thereof, and the like. The predetermined temperature preferably ranges from about 100° C. to about 320° C., alternatively from about 200° C. to about 300° C., and a pressure ranging from about 1.5 MPa to about 4.5 MPa, alternatively from about 2.0 MPa to about 4.0 MPa. The pure butyric acid product is preferably at least about 80 weight percent butyric acid, alternatively at least about 85 weight percent butyric acid, alternatively at least about 90 weight percent butyric acid, and alternatively at least about 95 weight percent butyric acid.

The liquid-phase continuous-flow reactor process encompasses several possible configurations, for example, trickle bed, fixed bed, or catalytic distillation. In the trickle bed reactor process, hydrogenation catalyst is packed into the bed. A liquid comprising maleic anhydride—which may contain the an alcohol or ester organic solvent such as ethanol, propanol, isopropanol, isobutanol, ethyl acetate, mixtures thereof, and the like—is fed into the top of the catalyst bed. The liquid then flows downward, and encounters an upward gas stream of hydrogen containing gas. In this manner, maleic anhydride is contacted hydrogen on the hydrogenation catalyst surface. A mixture of butyric acid, unreacted maleic anhydride, possible solvents, and by-products are collected at the bottom of the reactor. Butyric acid may then be separated and purified. The pure butyric acid product is preferably at least about 80 weight percent butyric acid, alternatively at least about 85 weight percent butyric acid, alternatively at least about 90 weight percent butyric acid, and alternatively at least about 95 weight percent butyric acid.

In an embodiment, the hydrogenation step may be carried out in a gas phase within a fixed-bed reactor or a fluid-bed reactor. In this embodiment, the hydrogenation may be performed under conditions ranging from about 180° C. to about 340° C. and from about 0 MPa to about 1.5 MPa. After purification, the pure bitric acid product is preferably at least about 80 weight percent butyric acid, alternatively at least about 85 weight percent butyric acid, alternatively at least about 90 weight percent butyric acid, and alternatively at least about 95 weight percent butyric acid.

The conversion of maleic anhydride to butyric acid in the liquid phase process ranges from about 80 to about 100 mole percent, alternatively from about 90 to about 100 mole percent, alternatively from about 95 to about 100 mole percent. The selectively of maleic anhydride to butyric acid in the liquid phase process ranges from about 70 to about 100 mole percent, alternatively from about 75 to about 100 mole percent, alternatively from about 80 to about 100 mole percent. The conversion of maleic anhydride to butyric acid in the gas phase process ranges from about 80 to about 100 mole percent, alternatively from about 85 to about 100 mole percent, alternatively from about 90 to about 100 mole percent. The selectivity of maleic anhydride to butyric acid in the gas phase process ranges from about 40 to about 80 mole percent, alternatively from about 40 to about 70 mole percent, alternatively from about 50 to about 60 mole percent.

To further illustrate various illustrative embodiments of the present invention, the following examples are provided, wherein Example 1 was conducted and Examples 2 through 5 are prophetic.

### EXAMPLES

### Example 1

This example demonstrates the first step of making maleic anhydride using a commercial VPO catalyst. A VPO catalyst was made according to U.S. Pat. No. 5,773,382, which is herein incorporated by reference in its entirety. Cylindrical tablets produced on the tableting press had a diameter of about 0.25 in., an average length of 0.200 in., and an average weight of 0.131 g. The Vox and P/V molar ratio were measured to be 4.21 and 1.071, respectively. The bulk density and surface area were 0.631 g/cc and 22.0 m²/g, respectively.

The performance of this catalyst was tested in a fixed bed maleic anhydride reactor at a standardized set of reaction conditions: 2.4±0.2 mole % n-butane in synthetic air (21 mol % oxygen/71 mol % helium, 103.4 kPa-g (15.0 psig) inlet pressure, and 1,500 GHSV. The catalyst (11.7 g) was charged to a 1.092 cm inside diameter×30.48 cm long (0.43 in. inside diameter by 1 ft. long) reactor to provide a catalyst bed of approximately 15.24 cm (6 in.) in length. The catalyst was rinsed for 138 hours. The reaction (bath temperature and maximum vield were determined when the catalyst was running at 85±2 mol % n-butane conversion.

The results showed a back calculated yield of 59.5% at n-butane conversion of 85.3%. The bath (reaction) temperature for this conversion was about 411° C.

### Example 2

This prophetic example demonstrates preparation of a catalyst for maleic anhydride hydrogenation to make butyric acid.

0.496 mol of acetic acid and 0.496 mol of titanium (IV) butoxide are added into 180 ml ethanol at 40° C. while stirring. A mixture composed of 22.4 ml of palladium chloride (1.36 mol) and 90 ml ethanol is drop-wise added into the above mixture while stirring. The addition is finished within 45 min. After completion of the addition, the final mixture is stirred for 1.5 hours and statically aged for 24 hours to obtain a gel. The gel is finally dried under supercritical fluid of ethanol at 260° C. and 8 MPa.

Based on a nitrogen isotherm at 77° K measured using micrometrics ASAP 2010, BET surface area and porosity of the catalyst are 105.2 m²/g and mean pore diameter is 8.0 nm. Pd dispersion is 0.92%, and particle size is about 7.3 nm based on TPR (temperature-programmed reduction) using 10% hydrogen in Ar with a ramp rate of 10° C./min from 25° C. to 600° C.

### Example 3

This prophetic example illustrates maleic anhydride hydrogenation to make butyric acid using the new catalyst made in Example 2.

To a 250 ml stainless steel autoclave charged with 25 ml ethanol, 7.85 grams of maleic anhydride and 1 gram of the catalyst made in Example 2 is added. After the reactor is purged with hydrogen three times, it is pressured up to 3 MPa and heated up to 240° C. When the pressure is steady, hydrogenation is initiated by stirring the mixture at 650 rpm.

After 2 hours of reaction, a liquid sample is analyzed by GC (gas chromatography) using a 20 m long capillary column with a diameter of 0.32 mm and flame ionization detector. The result shows 100% conversion of maleic anhydride and 94.3% selectivity to butyric acid. The only detected by-product is succinic anhydride with selectivity of 6.5%.

### Example 4

This is another prophetic example of catalyst preparation for maleic anhydride hydrogenation.

At room temperature, a mixture containing 23.8 grams of titanium (IV) butoxide and 60 grams of ethanol is drop-wise added into a suspension with 11.6 grams of γ-Al₂O₃ and 60 grams of de-ionized water while stirring. After stirring for 10 hours, the mixture is filtered and dried at 120° C. overnight to obtain powder. 6.58 grams of copper nitrate and 0.186 grams of palladium chloride are added to 20 grams of de-ionized water, and stirred. Ammonium hydroxide is added to adjust the pH value of the solution to 9. The above powder is added slowly into the solution with Pd and Cu while stirring. After stirring for 2 hours, the resulting product is dried at 120° C. for 24 hours and calcined in air at 500° C. for 4 hours to obtain a catalyst.

### Example 5

Catalytic performance of the catalyst prepared in Example 4 is presented in this prophetic example.

Hydrogenation of maleic anhydride is carried out in a continuous flow fixed bed reactor at atmospheric pressure. A quartz tube with an internal diameter of 12 mm is packed with 8 ml of the catalyst made in Example 4 and at both ends of the catalyst bed filled with quartz sands. The catalyst is in-situ reduced with 8% hydrogen in nitrogen at 160° C. for 8 hours and with 30% hydrogen in nitrogen at 360° C. for 4 hours.

20% (wt.) of maleic anhydride in propanol is pumped into the reactor, and maleic anhydride is vaporized in a quartz sand layer and mixed with hydrogen before contacting with catalyst. LHSV of maleic anhydride is 2.1 hour⁻¹, and GHSV of hydrogen is 260 hour⁻¹. The reaction temperature is kept at 275° C. After an hour reaction, the reaction product is collected for half an hour in an ice bath.

The collected sample is analyzed in the same manner as described in Example 3. The result shows 100% conversion of maleic anhydride and 58.3% selectivity to butyric acid.

Although the present invention and its advantages have been described in detail, it should be understood that various changes, substitutions and alterations may be made herein without departing from the spirit and scope of the invention as defined by the appended claims.

What is claimed is:

1. A process comprising:
combining a maleic anhydride, a hydrogen containing gas, and a hydrogenation catalyst to form butyric acid, wherein the selectivity of maleic anhydride to butyric acid is at least about 35 molar percent.

2. The process of claim 1, wherein the maleic anhydride is formed by combining one or more hydrocarbons and an oxygen gas selected from the group consisting of dioxygen, an oxygen containing gas, and mixtures thereof.

3. The process of claim 2, wherein the one or more hydrocarbons have at least four carbon atoms in a straight chain.

4. The process of claim 3, wherein the one or more hydrocarbons are selected from the group consisting of n-butane, n-butene, benzene, and mixtures thereof.

5. The process of claim 2, wherein the oxygen containing gas is air.

6. The process of claim 2, wherein a catalyst comprising vanadium, phosphorous, and oxygen is combined with the one or more hydrocarbons and the oxygen containing gas.

7. The process of claim 6, wherein the catalyst is either a vanadium phosphate oxide catalyst or a vanadium phosphate oxide catalyst coated with silicon.

8. The process of claim 7, wherein the catalyst further comprises additives selected from the group consisting of antimony, bismuth, boron, cerium, chromium, cobalt, copper, iron, lithium, molybdenum, nickel, niobium, silicon, tin, titanium, tungsten, uranium, zinc, zirconium, and mixtures thereof.

9. The process of claim 2, wherein the one or more hydrocarbons are present in any amount ranging from about 1.5 mole percent to about 2.5 mole percent, based on the total gas in the process.

10. The process of claim 2, wherein the one or more hydrocarbons are present in any amount ranging from about 1.0 molar percent to about 10.0 molar percent, based on the total gas in the process.

11. The process of claim 2, wherein the conversion of one or more hydrocarbon to maleic anhydride is any molar percent ranging from about 70 to about 90 percent.

12. The process of claim 2, wherein the yield of maleic anhydride ranges from about 40 to about 70 molar percent.

13. The process of claim 1, wherein the hydrogenation catalyst comprises at least one noble metal and at least one metal oxide, the at least one noble metal is selected from the group consisting of gold, platinum, palladium, rhodium, ruthenium, silver, tantalum, and mixtures thereof, the at least one metal oxide is selected from the group consisting of oxides of aluminum, chromium, iron, manganese, tin, titanium, vanadium, zirconium, and mixtures thereof.

14. The process of claim 1, wherein the hydrogenation catalyst comprises at least one transition metal and at least one metal oxide, the at least one transition metal is selected from the group consisting of chromium, cobalt, copper, hafnium, iron, magnesium, molybdenum, nickel, niobium, titanium, vanadium, zirconium, and mixtures thereof, the at least one metal oxide is selected from the group consisting of oxides of aluminum, chromium, iron, manganese, tin, titanium, vanadium, and zirconium.

15. The process of claim 1, wherein the hydrogenation catalyst comprises at least one noble metal, at least one transition metal and at least one metal oxide, the at least one noble metal is selected from the group consisting of gold, platinum, palladium, rhodium, ruthenium, silver, tantalum, and mixtures thereof, the at least one transition metal is selected from the group consisting of chromium, cobalt, copper, hafnium, iron, magnesium, molybdenum, nickel, niobium, titanium, vanadium, zirconium, and mixtures thereof, the at least one metal oxide is selected from the group consisting of oxides of aluminum, chromium, iron, manganese, tin, titanium, vanadium, and zircomum.

16. The process of claim 1, wherein the majority of the maleic anhydride and hydrogen containing gas are combined while in the liquid phase, and the hydrogenation catalyst comprises at least one noble supported on at least one metal oxide.

17. The process of claim 16, wherein the hydrogenation catalyst is Pd/TiO₂.

18. The process of claim 1, wherein the majority of the maleic anhydride and hydrogen containing gas are combined while in the gas phase, and the hydrogenation catalyst comprises at least one noble metal and at least one transition metal support on at least one metal oxide.

19. The process of claim 18, wherein the hydrogenation catalyst is Cu—Pd/TiO₂/γ-Al₂O₃.

20. The process of claim 1, wherein the hydrogenation catalyst comprises a noble metal in an amount less than about 5 weight percent based on the total weight of the hydrogenation catalyst.

21. The process of claim 16, wherein the conversion of maleic anhydride to butyric acid ranges from about 80 to about 100 molar percent.

22. The process of claim 16, wherein the selectively of maleic anhydride to butyric acid ranges from about 70 to about 100 molar percent.

23. The process of claim 18, wherein the conversion of maleic anhydride to buytric acid ranges from about 80 to about 100 molar percent.

24. The process of claim 18, wherein the selectively of maleic anhydride to butyric acid ranges from about 40 to about 80 molar percent.

25. A process of comprising:
(a) combining one or more hydrocarbons and an oxygen gas selected from the group consisting of dioxygen, an oxygen containing gas, and mixtures thereof to form maleic anhydride;
(b) selectively absorbing the maleic anhydride in an organic solvent selected from the group consisting of dibutyl phthalate (DBP), diisobutyl hexahydrophthalate, diisobutyl tetrahydrophthalate, dibutyl hexahydrophthalate, and dibutyl tetrahydrophthalate to form an absorption liquid;
(c) stripping the absorption liquid into a crude maleic anhydride, and optionally purifying the crude maleic anhydride into a maleic anhydride product;
(d) combining the maleic anhydride product and a second organic solvent selected from the group consisting of ethanol, propanol, isopropanol, isobutanol, ethyl acetate, and mixtures thereof to form a maleic anhydride solution, and optionally combining the crude maleic anhydride and a second organic solvent selected from the group consisting of ethanol, propanol, isopropanol, isobutanol, ethyl acetate, and mixtures thereof to form a maleic anhydride solution;
(e) heating the maleic anhydride solution to at least about 100° C.;
(f) combining the heated maleic anhydride solution, a hydrogen containing gas, and a hydrogenation catalyst to form a crude butyric acid, wherein the hydrogentation catalyst comprises at least one noble metal supported on at least one metal oxide, wherein the selectivity of maleic anhydride product to crude butyric acid is at least about 35 molar percent; and
(g) purifying the crude butyric acid into a butyric acid product, wherein the butyric acid product is at least about 80 weight percent butyric acid.
